(19) United States
(12) Patent Application Publication
(10) Pub. No.: US 2005/0170361 A1
(43) Pub. Date: Aug. 4, 2005

(54) Method of preparing reaction regions for biochips

(76) Inventors: Chih-Wei Ho, Taoyuan City (TW); Wen-Yeh Liao, Taipei (TW); Chien-Tsung Wu, Taichung (TW); Chao-Chi Pan, Hsinchu City (TW)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/769,960

(22) Filed: Feb. 2, 2004

(51) Int. Cl. C12Q 1/68; C12M 1/34; H01L 21/00

(52) U.S. Cl. 435/6; 435/287.2; 438/1

(57) ABSTRACT

A method of preparing reaction regions for biochips. First, a first member and a second member are provided as two carriers of biochips. At least one spacer is disposed between the first member and the second member to form a reaction region between the first member and the second member. Then, a sample solution is filled in the reaction regions.

### BACKGROUND OF THE INVENTION

1. Field of the Invention

The present invention relates to a reactor for a biochip, and particularly to a method of preparing reaction regions for two biochips disposed in parallel.

2. Description of the Related Art

Genetic information is essential to every manifestation of life, so many life science studies focus on developing methods to obtain the genetic information from living organisms. The information is useful for the subsequent studies such as discovery of disease-related genes and drug development.

In the past decade, the progress in decoding genetic codes of many living organisms has been exceptionally fruitful. For example, the decoding of whole human genomic sequences, which contains 3 billions DNA sequence information, was completed in April 2003. In the mean time, new technologies developed in other areas, such as optical electronics, micro-fabrications, and information technology, are being applied to make instrumentations that can facilitate life science research and medical applications. The combination of new technologies and genomic sequence information has been slowly transformed into a new generation of tools that allow scientists and researchers to obtain information about gene activities or genetic constituents of a large number of genes in a single experiment. Providing with the availability of the whole genome sequence, the capacity of these tools in theory should be able to cover the entire genome. This large number of experimental results is revolutionizing the medical and life science research because it provides unprecedented number of new leads for the subsequent applications in the fields of therapeutics and diagnostics. Biochips, or more specifically, microarrays, are one of such tools that have been developed for such application.

A microarray usually consists of a solid support (such as a glass slide, silicon wafer, and nylon- or polymer-based substrate) that contains numerous different reagents immobilized on the surface in a pre-arranged manner. These reagents (known as probes) are usually selected for their high specificity in binding affinity or reactivity toward their counterparts (known as targets) in biological samples. After applying a biological sample onto a microarray under an experimentally-controlled condition, the interactions between each probe on a microarray and its corresponding target in the biological sample can be observed through various target labeling techniques and appropriate detection instrumentation, thus providing the microarray user with qualitative and quantitative information about the target in the tested biological sample.

One type of microarray that has been used in a large extent is the DNA microarray. The DNA microarray uses DNA molecule or its derivatives as probes. These DNA probes bind to their targets in the biological sample (mostly cellular DNA or RNA fragments, or their derivatives) through the formation of double-helix based on the hydrogen-bonding between specific pairing of nucleic acids, a process known as DNA (or RNA) hybridization. With the availability of the whole genome sequences information, one can design a large number of DNA probes for a DNA microarray in order to obtain the experimental data that can cover all the genes in the genome. Therefore the amount of the experimental data that can be acquired from a DNA microarray experiment is now limited by the number of probes that can be physically included in a DNA microarray experiment with fixed amount of biological sample. The probe density of a microarray is mainly determined by its manufacturing method and a great amount of efforts have been directed into perfecting the manufacturing process by many microarray manufacturers. Through the advancement of the new technologies, such as micro-fabrication and precision machining, the density of the probes immobilized on the microarray can reach high, e.g., thousands of probes per square micron. However, to improve the probe density through the manufacturing process requires great amount of of time and resource for research and development and is time consuming.

Conventionally, the hybridization reaction can only be performed on one single microarray at a time. A popular but primitive method consists of applying the sample solution onto the microarray, covering the solution with a cover slip, and performing the hybridization in a humidified incubator. More elaborated methods, microarray packaging, or instruments have been developed to improve the handling of the sample solution or the consistency of the hybridization results. For example, U.S. Design Pat. 430,024 allow user to inject sample solution directly into a chamber between the microarray and a glass cover that were packaged together during the microarray manufacturing process. Another example, U.S. Pat. No. 6,485,918 disclosed a method and apparatus for incubation of a liquid reagent with target spots on a microarray substrate. According to U.S. Pat. No. 6,485,918, the apparatus has a deformable cover and a deflector. The deformable cover is adapted to seal the liquid reagent between the deformable cover and the surface of the microarray substrate, thus forming a reaction chamber. The deformable cover is then deformed by applying a force to the cover with the deflector. Thus, a reaction chamber for performing hybridization is formed between the liquid reagent and the substrate. However, in the method and apparatus disclosed in U.S. Pat. No. 6,485,918, a operator has to manually cover the deformable cover on the first surface of the microarray substrate to form the reaction chamber. This increases uncertainty and possible error in performing hybridization.

Another solution provides an apparatus or a device for automatically performing hybridization, such as U.S. Pat. Nos. 6,238,910 and 6,432,696. Both patents disclose a thermal and fluidic cycling device for nucleic acid hybridization, in which hybridization of nucleic acid samples is automatically performed. However, the apparatus disclosed in U.S. Pat. Nos. 6,238,910 and 6,432,696 is large-sized and expensive, which increases the cost of hybridization reactions. These methods or apparatuses do not address the issue of how to increase the capacity of a microarray experiment.

It is therefore necessary to provide an effective and economic way for performing hybridization reaction by preparing reaction regions for biochips. The new method disclosed in the present invention uses a simple modification on the conventional setups of biochip experiment that can also increase the capacity of a biochip experiment.

### SUMMARY OF THE INVENTION

Accordingly, it is an object of the present invention to provide a method of preparing reaction regions for biochips with a simple structure and reduced cost to provide consistent hybridization reactions from only a small amount of sample solution. According to the present invention, both the reaction regions and the capacity of the biochips can be increased.

It is another object of the present invention to provide a method of preparing reaction regions for biochips so that conditions of the two biochips with the same sample solution can be under control simultaneously.

The method disclosed in the present invention comprises assembling two biochips together with the probe-containing surface facing each other. Instead of placing a cover slip on a biochip to form a reaction region as described in the conventional method, the present invention replaces the cover slip with another biochip. The space formed between these two biochips serves as the reaction regions to accommodate a sample solution. In the method of the present invention, hybridization reaction performs on two biochips simultaneously with the same amount of target sample solution, compared to just one microarray done by the conventional method.

The present invention discloses a method of preparing reaction regions for biochips. According to the present invention, a first member and a second member are provided. At least one spacer is disposed between the first member and the second member to form a reaction region between the first member and the second member. Then, a sample solution is filled in the reaction region to form the reactor for biochips.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 is a perspective view showing an embodiment of a reactor for two biochips with different types of spacers according to the present invention;

FIG. 2 is a perspective view showing two biochips assembled by a holder; and

FIG. 3 is a perspective view showing another embodiment of the reactor for the biochips with a casing of the present invention.

### DETAILED DESCRIPTION OF THE INVENTION

Without intending to limit it in any manner, the present invention will be further illustrated by the following description. As will be appreciated by persons skilled in the art from the discussion herein, the present invention has a wide application in many industries. For discussion purposes, illustration is made herein to hybridization in biological technology. However, the present invention is not limited thereto.

The present invention discloses a method of preparing reaction regions for biochips. According to the present invention, both the reaction regions and the capacity of the biochips can be increased. The “biochips” include, but not limited to, gene chips, DNA chips, and microarrays. The “capacity” means the number of different types of probes for hybridization reaction. The “reaction region” means the space for hybridization reaction between two biochips with the probe-containing surface facing each other.

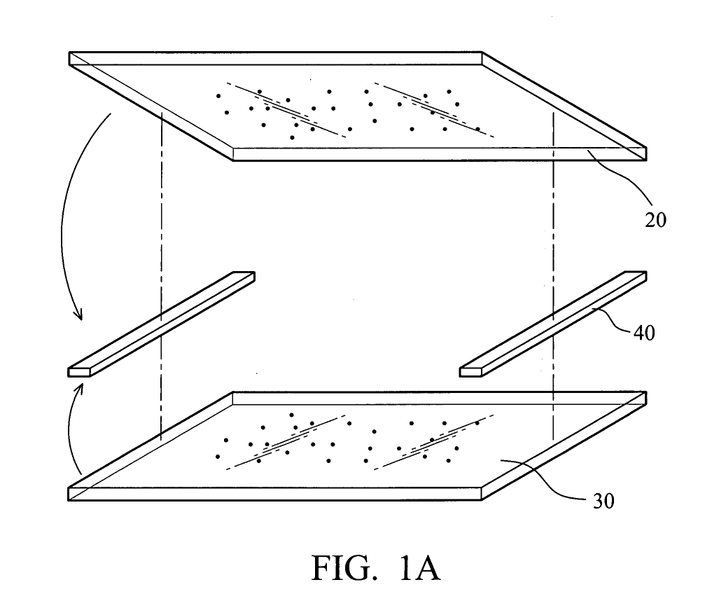

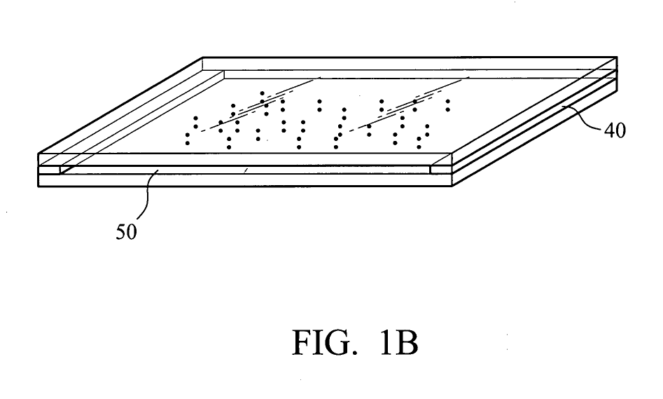

An embodiment of the reactor for biochips fabricated according to the present invention is described in detail with reference to FIGS. 1A, 1B, 1C and 1D.

Referring to FIG. 1A, the reactor of the embodiment comprises a first member 20 and a second member 30, which are provided as two carriers of biochips and can be composed of organic or inorganic materials. The first member 20 and the second member 30 are disposed in parallel to each other, and at least one spacer 40 (two spacers 40 in FIG. 1A) is disposed between the first member 20 and the second member 30. Due to the existence of the spacers 40, a reaction region 50 is formed between the first member 20 and the second member 30, as shown in FIG. 1B. Accordingly, a sample solution, which contains at least one type of molecule for sampling, can be provided to be filled in or sucked into the reaction region 50.

The molecule in the sample solution can be an organic molecule, an inorganic molecule or a biological molecule for performing hybridization reaction. The molecules can be charged or neutral. The organic molecules include, but are not limited to, organic acid, organic alkali, and amino acid. The inorganic molecules include, but are not limited to, metal ion and inorganic salt. The biological molecules include, but are not limited to, nucleic acid, oligonucleotide, protein, peptide and the derivatives thereof.

It should be noted that two spacers 40 are provided in the reactor of the above-mentioned embodiments, and the spacers 40 are bar-shaped. However, as shown in FIGS. 1C and 1D, there are several other types of spacers suitable in the present invention, e.g., spacers 41, 42. Size, shape and number of the spacers are not limited in the present invention.

According to the method of preparing reaction regions for biochips of the present invention, a simple structure of the reactor for biochips can be obtained. Since two biochips are formed in the reactor, both the reaction regions and the capacity for utilization can be increased.

Further, the first member 20 and the second member 30, which serve as two biochips, can be two identical biochips with the same reaction regions so that reactions can be duplicated. On the other hand, the first member 20 and the second member 30 can be two different biochips, such as different biochips for gene verification, or two compensated biochips, such as two biochips for human gene identification, in which one biochip is for a portion of human genes, and the other biochip is for the remaining portion of human genes. Or the first biochip is for one tissue and the second biochip is for another tissue, for gene expression assay. The first member 20 and the second member 30 can be another type of two different biochips, for example, biochips for two different species, in which one biochip is for human gene, and the other biochip is for other species, rodent, for example.

Further, the reactor fabricated according to the present invention provides consistent hybridization reactions from a small amount of sample solution with reduced cost, so that uncertainty and possibility of error in performance of hybridization can be greatly reduced.

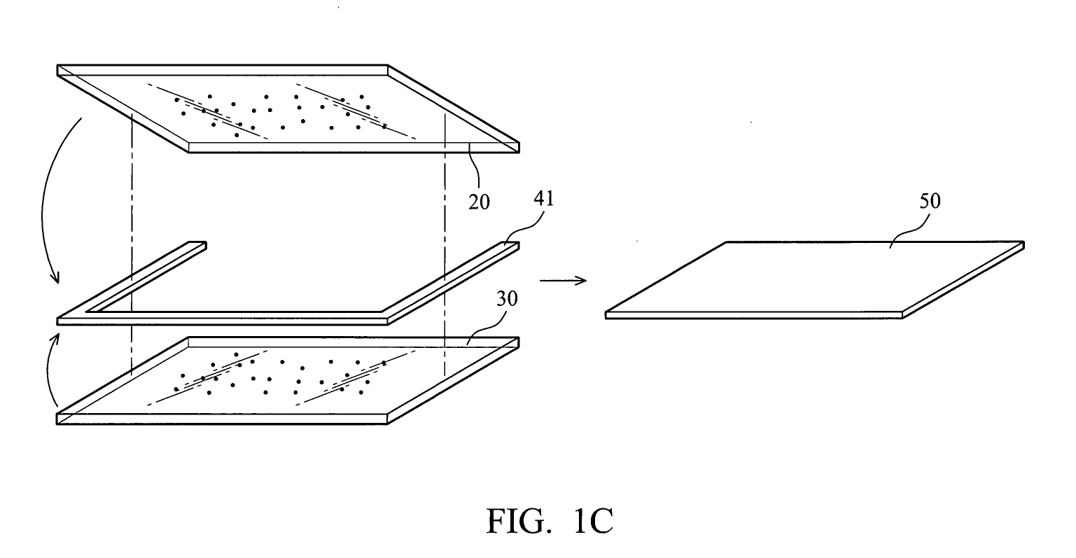

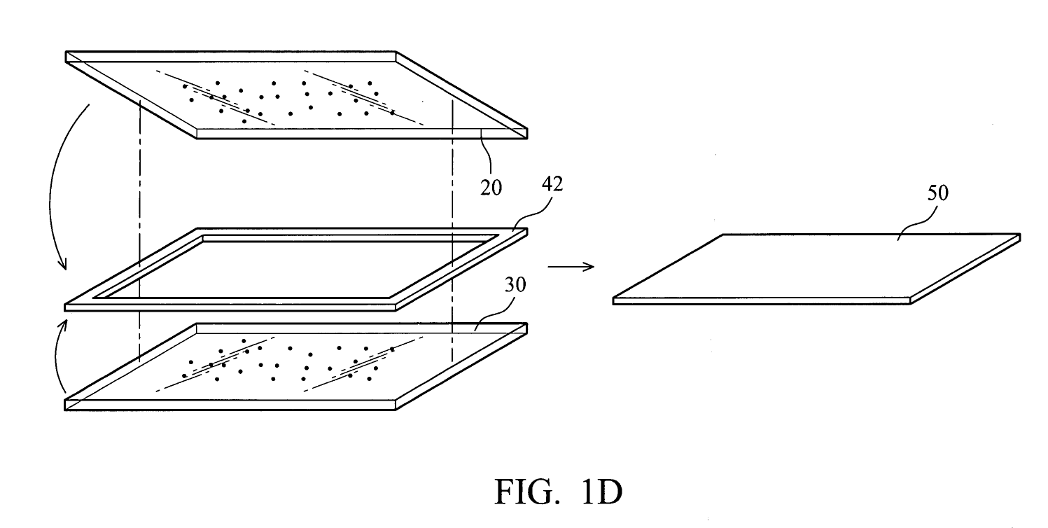

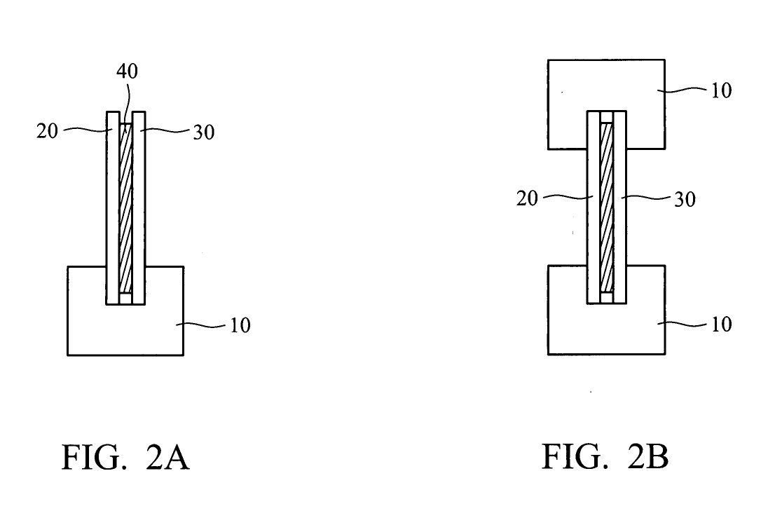

Referring to FIG. 2, another preferred embodiment, the reactor for a biochip according to the present invention comprises a first member 20, a second member 30, and at least one spacer 40 (e.g. two spacers) disposed between the first member 20 and the second member 30. The reactor further comprises at least one holder 10. The holder 10 assembles the first member 20 and the second member 30, and can maintain the reaction region 50 between the two members.

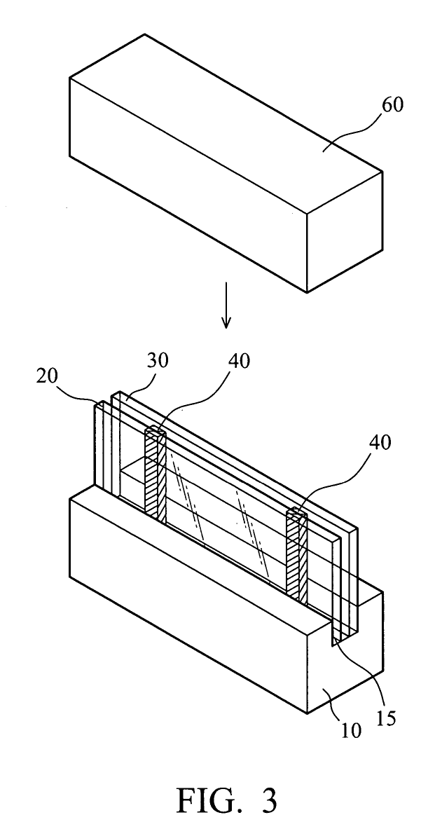

In another preferred embodiment of the reactor for a biochip according to the present invention comprises a first member 20, a second member 30, at least one spacer 40 (e.g. two spacers) disposed between the first member 20 and the second member 30, a holder 10, and a casing 60, as shown in FIG. 3. The casing 60 is provided to cover the holder 10 to enclose the hybridization space 50 in a sealed environment. Accordingly, hybridization can be performed in the sealed environment.

While the invention has been described, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

1. A method of preparing reaction regions for biochips, comprising the steps of:
providing a first member and a second member;
disposing at least one spacer between the first member and the second member to form a reaction region between the first member and the second member; and
filling a sample solution in the reaction region.

2. The method of preparing reaction regions for biochips as claimed in claim 1, wherein the first member and the second member are biochips.

3. The method of preparing reaction regions for biochips as claimed in claim 2, wherein said biochips are the same.

4. The method of preparing reaction regions for biochips as claimed in claim 2, wherein said biochips are different.

5. The method of preparing reaction regions for biochips as claimed in claim 1, further comprising a holder to assemble the first member and the second member.

6. The method of preparing reaction regions for biochips as claimed in claim 1, further comprising enclosing the reaction regions in a sealed environment.

7. The method of preparing reaction regions for biochips as claimed in claim 1, wherein the first member and the second member are inert to the sample solution.

8. The method of preparing reaction regions for biochips as claimed in claim 1, further comprising a step of incubating the reaction region under hybridization condition.

9. An apparatus containing reaction regions for biochips, comprising:
a first member and a second member disposed in parallel; and
at least one spacer disposed between the first member and the second member to form a reaction region between the first member and the second member.

10. The apparatus as claimed in claim 9, wherein the first member and the second member are biochips.

11. The apparatus as claimed in claim 10, wherein said biochips are the same.

12. The apparatus as claimed in claim 10, wherein said biochips are different.

13. The apparatus as claimed in claim 9, further comprising a holder to assemble the first member and the second member.

14. The apparatus as claimed in claim 9, wherein the reaction regions are enclosed in a sealed environment.
